(19) United States
(12) Patent Application Publication
(10) Pub. No.: US 2024/0023853 A1
(43) Pub. Date: Jan. 25, 2024

(54) Blood Collection Set and Related Systems and Methods

(71) Applicant: Becton, Dickinson and Company, Franklin Lakes, NJ (US)

(72) Inventors: Olivia HU, Shanghai (CN); Simon Liu, Shanghai (CN); Bo Yan, Shanghai (CN)

(21) Appl. No.: 18/042,028

(22) PCT Filed: May 5, 2021
(86) PCT No.: PCT/US21/30921

§ 371 Date: Feb. 17, 2023

(30) Foreign Application Priority Data
Aug. 21, 2020 (CN) 202010847358.3
Aug. 21, 2020 (CN) 202021770749.1

(51) Int. Cl. A61B 5/15; A61M 39/10

(52) U.S. Cl. CPC A61B 5/150267; A61B 5/150992; A61M 39/10; A61M 39/105; A61M 2039/1077; A61M 2039/1072

(57) ABSTRACT

A blood collection set to couple to a catheter system may include a needle assembly and/or a housing. The needle assembly may include a body, a sharp needle extending proximally from the body, and a sheath covering a proximal tip of the sharp needle. The housing may be coupled to a distal end of the needle assembly. The housing may be configured to create a fluid path through a connector of the catheter system.

### BACKGROUND

Catheters are generally used for parenteral nutrition, intravenous fluid replacement, and administering analgesics and antibiotics. Catheters are also used for blood draw. Catheters can be inserted at the bedside using sterile techniques and can remain in place for several weeks.

A common type catheter is an over-the-needle catheter. As its name implies, a catheter that is “over-the-needle” may be mounted over an introducer needle having a sharp distal tip. The sharp distal tip may be used to pierce skin and a vein of a patient. Insertion of the catheter into the vein may follow the piercing of the vein by the introducer needle. The introducer needle typically has the sharp distal tip to pierce skin and the vein of the patient with minimal resistance to minimize the pain to the patient.

The introducer needle is generally placed at a steep inclined angle with respect to a surface of the skin and a longitudinal dimension of the vein to be pierced to allow penetration through the skin and a wall of the vein. The needle and the catheter are generally inserted with a bevel of the introducer needle facing away from the skin of the patient. After the tip of the introducer needle pierces the wall, the angle of the insertion is lowered to be able to slide the introducer needle and the catheter into the vein a distance to position the catheter securely in the vein.

Once placement of the introducer needle within the vein has been confirmed, the introducer needle may be withdrawn and removed, leaving the catheter in place for future fluid infusion and/or blood withdrawal. After blood withdrawal through the catheter, a catheter system, including the catheter, may be flushed to remove residual blood. The catheter system may then be used for infusion. This process, however, can be both time-consuming and hazardous as multiple steps are required to perform blood collection and infusion. Inherent in each process step is a risk of contamination from residual blood. Also, flushing processes to remove residual blood from the catheter system tend to be both time-consuming and unreliable, as grooves and recesses of components of the catheter system tend to retain residual blood and/or fluid even after flushing.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a blood collection set and related devices, systems, and methods. In some embodiments, the blood collection set may be coupled to a catheter system. In some embodiments, the catheter system may include a catheter adapter, which may include a distal end, a proximal end, and a lumen extending through the distal end of the catheter adapter and the proximal end of the catheter adapter. In some embodiments, the catheter system may include a catheter extending distally from the distal end of the catheter adapter. In some embodiments, the catheter may include a peripheral intravenous catheter, a peripherally-inserted central catheter, or a midline catheter.

In some embodiments, the catheter system may include a connector, which may be coupled to the catheter adapter. In some embodiments, the connector may include a septum. In some embodiments, the septum may include a slit. In other embodiments, the septum may not include a slit.

In some embodiments, the blood collection set may include a needle assembly and/or a housing. In some embodiments, the needle assembly may include a body, a sharp needle extending proximally from the body, and a sheath covering a proximal tip of the sharp needle. In some embodiments, the housing may be coupled to the connector. In some embodiments, the housing may be configured to create the fluid path through the connector.

In some embodiments, the housing may be directly and removably coupled to the needle assembly. In these and other embodiments, the blood collection set may be used for infusion after blood collection via the needle assembly and removal of the needle assembly from the housing. In some embodiments, the housing may be integrated with the needle assembly. In further detail, the housing may be permanently coupled to the needle assembly or monolithically formed with the body of the needle assembly as a single unit.

In some embodiments, the catheter system may include a side port disposed between the distal end of the catheter adapter and the proximal end of the catheter adapter. In some embodiments, the catheter system may include an extension tube. In some embodiments, the catheter system may include an adapter, which may include a distal end and a port. In some embodiments, the adapter may include a Y-adapter, a straight adapter, or another suitable adapter. In some embodiments, the extension tube may include a distal end integrated with the side port of the catheter adapter and/or a proximal end integrated with the distal end of the adapter.

In some embodiments, the connector may include a needleless connector. In some embodiments, the housing may be disposed in a proximal position. In some embodiments, the housing may include a male luer. In some embodiments, the male luer may be configured to activate the septum and create the fluid path through the connector in response to the housing being moved from the proximal position to a distal position. In some embodiments, the housing may be threaded from the proximal position to the distal position or pushed from the proximal position to the distal position.

In some embodiments, the housing may include a distal piece and a proximal piece movable distally towards the distal piece. In some embodiments, the distal piece of the housing may include a male luer. In some embodiments, the male luer may be configured to activate the septum and create the fluid path through the connector in response to the proximal piece moving distally towards the distal piece.

In some embodiments, the housing may include a cannula configured to penetrate the septum of the connector to create the fluid path through the connector. In some embodiments, the cannula may extend through the septum. In some embodiments, the cannula may include one or more side holes in fluid communication with a lumen of the cannula. In some embodiments, the housing may be movable from a proximal position to a distal position. In some embodiments, in response to the housing moving from the proximal position to the distal position, the cannula may extend through the septum and the fluid path through the connector may be created.

In some embodiments, the catheter system may include an extension set, which may include a distal end coupled to the proximal end of the catheter adapter and/or a proximal end coupled to the connector. In these and other embodiments, the connector may include the needleless connector.

In some embodiments, a system may include the extension set. In some embodiments, the distal end of the extension set may be configured to couple to the proximal end of the catheter adapter. In some embodiments, the system may include the needle assembly. In some embodiments, the needle assembly may be directly coupled to the proximal end of the extension set. In some embodiments, the system may include the catheter system. In some embodiments, the system may include the catheter adapter and a catheter extending distally from the distal end of the catheter adapter. In some embodiments, the system may include the needleless connector directly coupled to the proximal end of the extension set. In some embodiments, the system may include the housing, which may be directly coupled to the needleless connector and the needle assembly. In some embodiments, the housing may be configured to create a fluid path through the needleless connector.

In some embodiments, a method may include inserting a catheter of a catheter system into vasculature of a patient. In some embodiments, the method may include coupling the blood collection set to the catheter system. In some embodiments, the housing may include a male luer. In some embodiments, the housing may be disposed in a proximal position, and the method may include moving the housing from the proximal position to a distal position. In some embodiments, the male luer may activate the septum and create the fluid path through the connector in response to the housing being moved from the proximal position to the distal position. In some embodiments, the housing may be threaded from the proximal position to the distal position or pushed from the proximal position to the distal position. In some embodiments, the method may further include collecting blood from a patient after moving the housing from the proximal position to the distal position.

In some embodiments, the housing may include the distal piece and the proximal piece movable distally towards the distal piece. In some embodiments, the distal piece of the housing may include a male luer. In some embodiments, the method may include moving the proximal piece distally towards the distal piece. In some embodiments, the male luer may activate the septum and create the fluid path through the connector in response to the proximal piece moving distally towards the distal piece. In some embodiments, the method may further include collecting blood from the patient after moving the proximal piece towards the distal piece.

In some embodiments, the housing may include the cannula, and the cannula may extend through the septum in response to coupling the blood collection set to the catheter system. In some embodiments, the method may include collecting blood from the patient through the blood collection set.

In some embodiments, the housing may include the cannula, and the method may include moving the housing from a proximal position to a distal position. In some embodiments, in response to the housing moving from the proximal position to the distal position, the cannula may extend through the septum and the fluid path through the connector may be created. In some embodiments, the method may further include collecting blood from a patient after moving the housing from the proximal position to the distal position.

In some embodiments, a method may include coupling an extension set to the proximal end of the catheter adapter. In some embodiments, the extension set may include a distal end and a proximal end. In some embodiments, the method may include coupling the distal end of the extension set to the proximal end of the catheter adapter. In some embodiments, the method may include coupling the needle assembly to the proximal end of the extension set and drawing blood from a patient. In some embodiments, the method may include coupling the connector to the proximal end of the extension set and/or coupling the blood collection set to the connector. In some embodiments, the method may further include collecting blood through the extension set.

In some embodiments, the method may include removing the needle assembly from the blood collection set coupled to the catheter system, and after removing the needle assembly from the blood collection set, flushing the catheter system or infusing fluid through the catheter system.

As used in the present disclosure, the term “coupling” may include, but is not limited to, direct coupling. It is to be understood that both the foregoing general description and the following detailed description are examples and explanatory and are not restrictive of the present disclosure. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example catheter system, according to some embodiments;

FIG. 1B is an upper perspective view of an example blood collection set, according to some embodiments;

FIG. 1C is a cross-sectional view of the blood collection set, according to some embodiments;

FIG. 1D is another cross-sectional view of the blood collection set, according to some embodiments;

FIG. 2A is an upper perspective view of the catheter system, illustrating coupling of an example blood collection tube to the catheter system, according to some embodiments;

FIG. 2B is a cross-sectional view of the blood collection set coupled to an example connector, according to some embodiments;

FIG. 3 is an upper perspective view of the catheter system, illustrating an engaged example pinch clamp and an example needle assembly of the blood collection set removed, according to some embodiments;

FIG. 4 is an upper perspective view of an example adapter coupled to the connector and an example housing of the blood collection set, according to some embodiments;

FIG. 5 is an upper perspective view of the catheter system coupled to an example syringe, according to some embodiments;

FIG. 6A is a side view of the connector, according to some embodiments;

FIG. 6B is a cross-sectional view of the connector, according to some embodiments;

FIG. 7 is a cross-sectional view of a portion of the catheter system coupled to the syringe, according to some embodiments;

FIG. 8 is an upper perspective view of a portion of the catheter system coupled to an example infusion set, according to some embodiments;

FIG. 9 is an upper perspective view of the catheter system, illustrating removal of the housing, according to some embodiments;

FIG. 10 is an enlarged perspective view of the housing of the blood collection set, according to some embodiments;

FIG. 11 is an upper perspective view of a portion of the catheter system, illustrating removal of another example needle assembly and an example blood control septum, according to some embodiments;

FIG. 12A is an enlarged perspective view of a portion of the catheter system, illustrating the blood collection set disposed in an example proximal position, according to some embodiments;

FIG. 12B is an enlarged perspective view of the catheter system, illustrating the blood collection set disposed in an example distal position, according to some embodiments;

FIG. 13A is an upper perspective view of an example needleless connector, according to some embodiments;

FIG. 13B is an upper perspective view of another example needleless connector, according to some embodiments;

FIG. 13C is an upper perspective view of another example blood collection set configured to couple to a needleless connector, according to some embodiments;

FIG. 13D is a cross-sectional view of the blood collection set of FIG. 13C, according to some embodiments;

FIG. 13E is an upper perspective view of the blood collection set of FIG. 13C, illustrating another example housing removed from the needle assembly, according to some embodiments;

FIG. 14A is a side view of another example housing of the blood collection set of FIG. 13C, illustrating a luer lock, according to some embodiments;

FIG. 14B is a side view of the housing of FIG. 14A coupled to the needle assembly, illustrating a luer slip, according to some embodiments;

FIG. 14C is an enlarged perspective view of the luer slip coupled to the needleless connector, according to some embodiments;

FIG. 14D is a cross-sectional view of the blood collection set of FIG. 13C coupled to the needleless connector of FIG. 13A, illustrating the blood collection set in an example proximal position, according to some embodiments,

FIG. 14E is a cross-sectional view of the blood collection set of FIG. 13C coupled to the needleless connector of FIG. 13A, illustrating the blood collection set in an example distal position, according to some embodiments,

FIG. 15A is an upper perspective view of the catheter system, illustrating another example blood collection set, according to some embodiments;

FIG. 15B is a perspective view of the blood collection set of FIG. 15A, illustrating an example distal piece in a first position, according to some embodiments;

FIG. 15C is a cross-sectional view of the blood collection set of FIG. 15A coupled to an example needleless connector, illustrating the distal piece in an example first position, according to some embodiments;

FIG. 15D is a perspective view of the blood collection set of FIG. 15A, illustrating the distal piece in an example second position, according to some embodiments;

FIG. 15E is a cross-sectional view of the blood collection set of FIG. 15A, illustrating the distal piece in the second position, according to some embodiments;

FIG. 16 is an upper perspective view of the catheter system, illustrating an example adapter having an example injection port, according to some embodiments;

FIG. 17A is an enlarged perspective view of the injection port of FIG. 16, illustrating an example cap, according to some embodiments;

FIG. 17B is an enlarged perspective view of the injection port of FIG. 16 illustrating the cap opened, according to some embodiments;

FIG. 18 is an upper perspective view of the catheter system, illustrating an example straight adapter, according to some embodiments;

FIG. 19 is an enlarged perspective view of an example elongate end cap coupled to an example adapter, according to some embodiments;

FIG. 20 is an upper perspective view of the catheter system, illustrating an example secondary flashback chamber, according to some embodiments;

FIG. 21A is an enlarged top view of an example cannula tip, according to some embodiments;

FIG. 21B is an enlarged upper perspective view of the cannula tip of FIG. 21A, according to some embodiments;

FIG. 22A is an enlarged top view of another example cannula tip, according to some embodiments;

FIG. 22B is an enlarged upper perspective view of the cannula tip of FIG. 22A, according to some embodiments;

FIG. 23A is an upper perspective view of the catheter system, illustrating an example extension set coupled to the blood collection set, according to some embodiments;

FIG. 23B is an upper perspective view of the catheter system, illustrating the extension set coupled to the blood collection set of FIG. 13C, according to some embodiments;

FIG. 23C is an upper perspective view of the catheter system, illustrating the extension set coupled to the blood collection set of FIG. 15A, according to some embodiments; and

FIG. 24 is an upper perspective view of the catheter system, illustrating the extension set coupled to a proximal end of an example catheter adapter, according to some embodiments.

### DESCRIPTION OF EMBODIMENTS

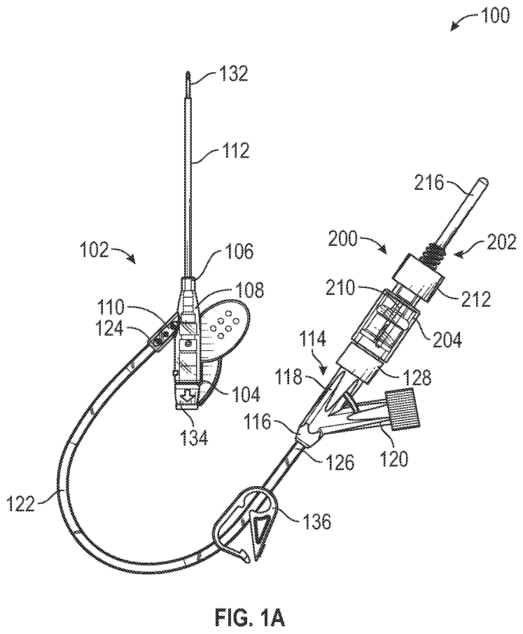

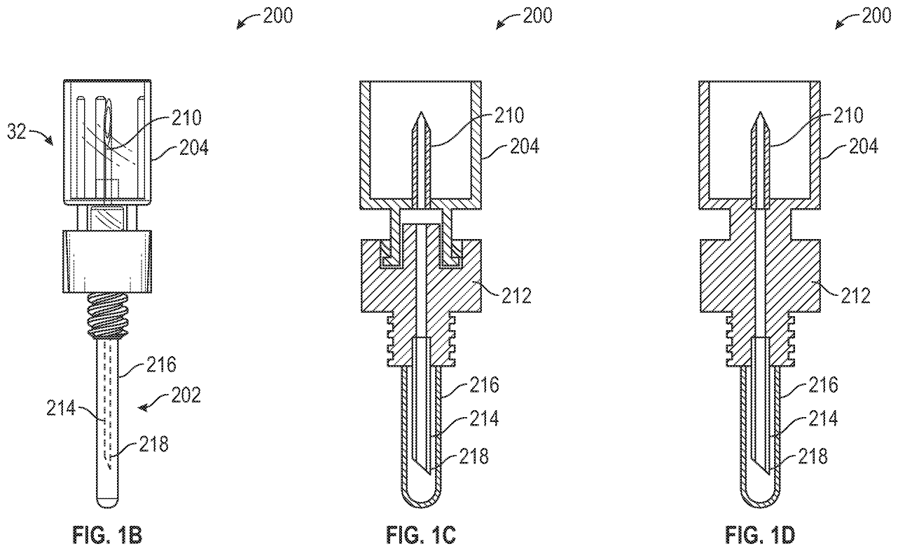

Referring now to FIG. 1, in some embodiments, a catheter system 100 may include a catheter adapter 102, which may include a proximal end 104, a distal end 106, and a lumen 108 extending therebetween. In some embodiments, a side port 110 may be disposed between the distal end 106 of the catheter adapter 102 and the proximal end 104 of the catheter adapter 102. In these and other embodiments, a catheter 112 may extend distally from the distal end 106 of the catheter adapter 102.

In some embodiments, the catheter 112 may include a peripheral intravenous catheter, a peripherally-inserted central catheter, or a midline catheter. In some embodiments, a needle hub 134 may be removably coupled to the proximal end 104 of the catheter adapter 102. In some embodiments, the catheter 112 may include an over-the-needle catheter such that an introducer needle 132 may extend from the needle hub 134 and through the catheter 112 to insert the catheter system 100 into the vasculature of the patient. In some embodiments, the catheter 112 and the introducer needle 132 may be assembled such that the sharp proximal tip of the introducer needle 132 extends beyond the distal tip of the catheter 112, with the bevel of the introducer needle 132 facing up away from skin of the patient.

In some embodiments, the introducer needle 132 and the catheter 112 may be inserted at a shallow angle through the skin into the vasculature of the patient. In some embodiments, in response to the introducer needle 132 and the catheter 112 being inserted into the vasculature of the patient, blood may flow through the introducer needle 132 and into the needle hub 134, and the blood may be visible within the needle hub 134 to indicate to a user that the introducer needle 132 is within the vasculature of the patient.

In some embodiments, the catheter system 100 may include an adapter 114. In some embodiments, the adapter 114 may include a Y-adapter, a straight adapter, or another suitable adapter. In some embodiments, the adapter 114 may include one or more of a distal end 116, a first port 118, and a second port 120. In some embodiments, the adapter 114 may include a reduced volume and/or reduced dimensions relative to a standard adapter to avoid blood contamination. In some embodiments, in response to the catheter 112 being inserted into the vasculature of the patient, blood may flow through the catheter 112, at least a portion of the catheter adapter 102, the extension tube 122, and into the adapter 114.

In some embodiments, a port of the adapter 114 may be coupled to a connector 128. In some embodiments, the first port 118 and/or the second port 120 of the adapter 114 may be coupled to the connector 128. In further detail, in some embodiments, the connector 128 may be monolithically formed as a single unit with the first port 118 and/or second port 120. In other embodiments, the first port 118 and/or the second port 120 of the adapter 114 may be coupled to the connector 128 via a luer connection. In these embodiments, the connector 128 may include a luer, such as, for example, a female or male luer. In some embodiments, the luer may be threaded or slip-fit with the adapter 114.

In some embodiments, the catheter system 100 may include the extension tube 122, which may include a distal end 124 integrated with the side port 110 and a proximal end 126 integrated with the distal end 116 of the adapter 114. In some embodiments, a clamp, such as, for example, a pinch clamp 136 or another suitable clamp, may be disposed on the extension tube 122 and configured to selectively clamp the extension tube 122 to close a fluid path through the extension tube 122. In some embodiments, the pinch clamp 136 may be replaced with any suitable clamp.

In some embodiments, the connector 128 may include a proximal end configured to receive a blood collection set 200. Some embodiments of the blood collection set 200 may include a needle assembly 202 and a housing 204, which may be configured to engage the connector 128. In some embodiments, the housing 204 may be cylindrical. In some embodiments, the blood collection set 200 may be used for one or more of collecting blood from a patient, flushing, and infusion.

Referring now to FIGS. 1A-1C, in some embodiments, the needle assembly 202 of the blood collection set 200 may include a body 212, a sharp needle 214 extending proximally from the body 212, and a sheath 216 covering a sharp proximal tip 218 of the sharp needle 214. In some embodiments, the sheath 216 may be elastomeric. In some embodiments, the sheath 216 may be plastic or another suitable material. In some embodiments, the sharp needle 214 may be secured within the body 212 via a press fit, an adhesive, a mechanical device, or another suitable mechanism. In some embodiments, the sharp needle 214 may extend proximally beyond the body 212.

In some embodiments, the sheath 216 may be coupled to the body 212 of the needle assembly 202. In some embodiments, the sharp proximal tip 218 of the sharp needle 214 may be enveloped within the sheath 216. In some embodiments, the sheath 216 may include an open distal end and a closed proximal end.

Referring now to FIG. 1C, in some embodiments, the housing 204 and the body 212 may be removably coupled together and separable. In these and other embodiments, the blood collection set 200 may be used for infusion after blood collection via the needle assembly 202 and removal of the needle assembly 202 from the housing 204. Referring now to FIG. 1D, the housing 204 and the body 212 of the needle assembly 202 may be integrated or monolithically formed as a single unit.

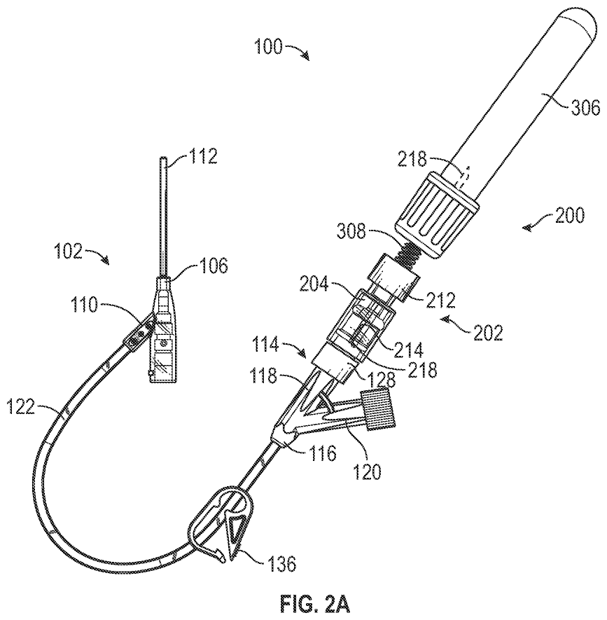

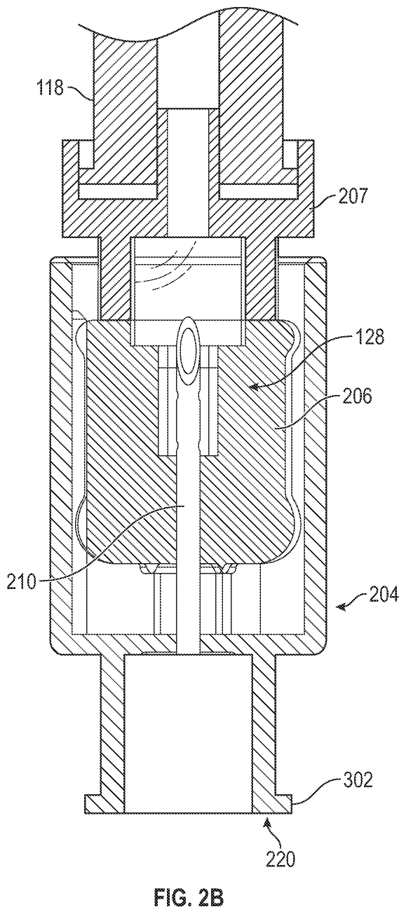

Referring now to FIG. 2A, in some embodiments, the housing 204 and the blood collection set 200 may be pre-attached to the connector 128 and the catheter system 100. In some embodiments, the catheter system 100 may be assembled and provided with the housing 204 pre-attached to the connector 128. In other embodiments, the user may couple the blood collection set 200 to the connector 128. In some embodiments, the blood collection set 200 may be configured to receive a blood collection device 306 such as a blood collection tube, for example. In some embodiments, in response to the blood collection device 306 pushing the sheath 216 distally towards the body 212, the sharp needle 214 may pierce the sheath 216 and the sharp proximal tip 218 of the needle assembly 202 may be inserted into the blood collection device 306.

In some embodiments, inserting the sharp proximal tip 218 into the blood collection device 306 may couple the blood collection device 306 to the blood collection set 200. In some embodiments, the blood collection device 306 may be coupled to the blood collection set 200 after the catheter 112 is positioned within the vasculature. In some embodiments, when the blood collection device 306 is penetrated by the sharp proximal tip 218, blood may flow into the blood collection device 306 due to a pressure differential between the vasculature and the blood collection device 306. In some embodiments, the needle assembly 202 may include threading 308, which may be coupled to corresponding threading of a cylindrical holder (not illustrated) for the blood collection device 306.

Referring now to FIG. 2B, in some embodiments, a proximal end of the connector 128 may include a septum 206, which may be coupled to a body 207 of the connector 128. In some embodiments, the body 207 may be rigid or semi-rigid. In some embodiments, the septum 206 may be constructed of an elastomeric and/or resilient material. In these and other embodiments, the septum 206 may include a slit or may not include the slit.

In some embodiments, the housing 204 may include a cannula 210 configured to penetrate or extend through the septum 206 to provide a fluid path through the connector 128. In these and other embodiments, a tip of the cannula 210 may be disposed within the housing 204, which may decrease a risk of injury to a user. In some embodiments, the tip of the cannula 210 may be blunt, which may decrease a risk of injury to the connector 128, or sharp, which may facilitate penetration of the septum 206. In some embodiments, the cannula 210 may extend distally from the housing 204, and a proximal end of the cannula 210 may be secured within the housing 204 via a press fit, an adhesive, a mechanical device, or another suitable mechanism.

In some embodiments, the cannula 210 may be constructed of metal or another suitable material. In some embodiments, an outer diameter of the cannula 210 may be uniform along all or a portion of a length of the cannula 210. In some embodiments, a portion of the housing 204 from which the cannula 210 extends may be constructed of plastic or another suitable material. In some embodiments, the proximal end 220 and/or a cylindrical portion of the housing 204 surrounding the cannula 210 may be constructed of plastic or another suitable material.

In some embodiments, a proximal end 220 of the housing 204 may include a luer 302 configured to couple to a corresponding luer of the needle assembly 202. Some embodiments of the luer 302 may include, for example, a female luer or a male luer. In some embodiments, the luer 302 may be threaded or slip-fit.

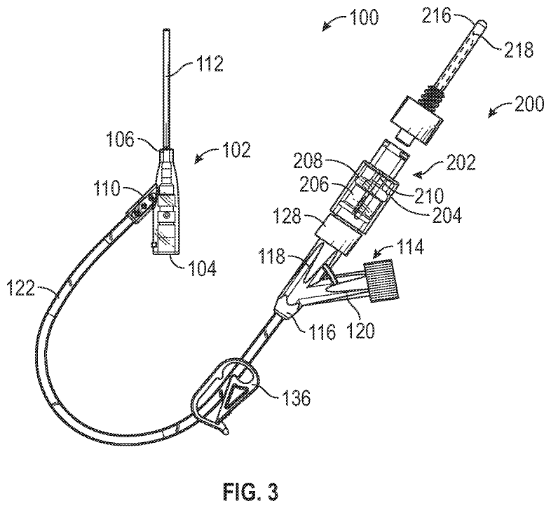

Referring now to FIG. 3, in some embodiments, prior to removing the cannula 210 from the septum 206 and the needle assembly from the catheter system 100, the user may first engage the pinch clamp 136 disposed on the extension tube 122 to stop fluid flow through a fluid path of the catheter system 100, which may extend from the catheter 112, through the extension tube 122, through the adapter 114, through the connector 128, through the cannula 210, and into the needle assembly 202. In some embodiments, the needle assembly 202 may be removed from the catheter system 100 after blood is collected in the blood collection device 306 and/or the pinch clamp 136 is engaged. In these and other embodiments, the housing 204 and the blood collection set 200 may be pre-attached to the connector 128 and the catheter system 100. In some embodiments, the catheter system 100 may be assembled and provided with the housing 204 pre-attached to the connector 128.

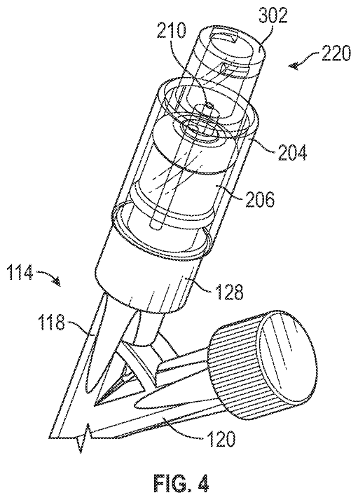

Referring now to FIG. 4, the luer 302 of the proximal end 220 of the housing 204 is illustrated, according to some embodiments. In some embodiments, the needle assembly 202 (see, for example, FIG. 3) may include a luer, such as, for example, a male luer, to couple to the luer 302.

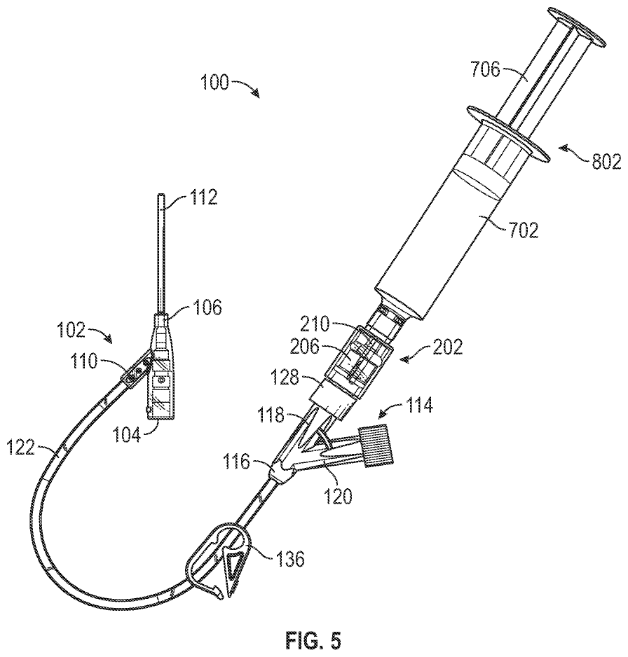

Referring now to FIG. 5, in some embodiments, the needle assembly 202 may be uncoupled from the housing 204, and then a syringe 802 may be coupled to the housing 204. In operation, in some embodiments, the pinch clamp 136 may be disengaged from the extension tube 122 to open the fluid path through the catheter system 100. In some embodiments, the syringe 802 may be coupled to the connector 128 by a threaded luer, a luer-slip, a press fit, etc. In some embodiments, the syringe 802 may be used for flush or infusion after collecting blood and removal of the needle assembly 202. In these and other embodiments, the housing 204 and the blood collection set 200 may be pre-attached to the connector 128 and the catheter system 100. In some embodiments, the catheter system 100 may be assembled and provided with the housing 204 pre-attached to the connector 128.

In some embodiments, the syringe 802 may be filled with fluid 702 such as saline or another suitable fluid to remove residual blood after blood collection. In some embodiments, the syringe 802 may be actuated by depressing a plunger 706 of the syringe 802 to release the fluid from the syringe 802 and flush the catheter system 100.

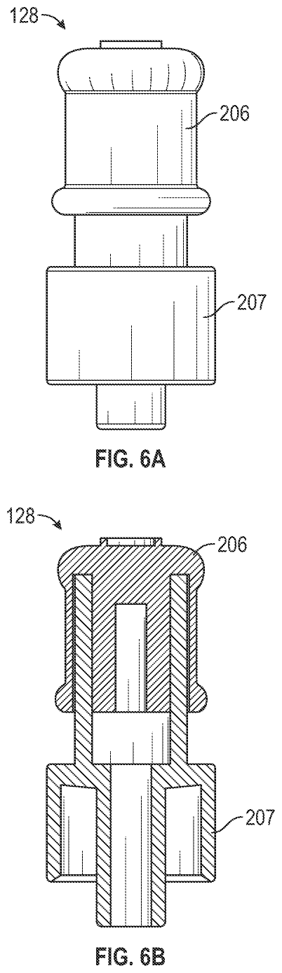

Referring now to FIGS. 6A-6B, the proximal end of the connector 128 may include the septum 206, which may be coupled to the body 207 of the connector 128. In some embodiments, the body 207 may be rigid or semi-rigid. In some embodiments, the septum 206 may be constructed of an elastomeric and/or resilient material. In these and other embodiments, the septum 206 may include a slit or may not include the slit. In some embodiments, the cannula 210 (see, for example, FIGS. 1-5) may penetrate or extend through the septum 206 to provide the fluid path through the connector 128.

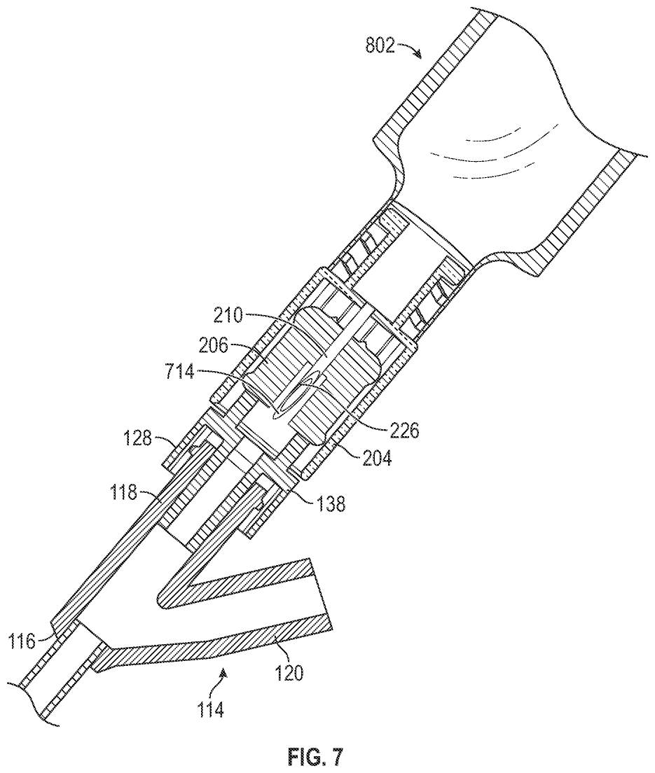

Referring now to FIG. 7, in some embodiments, the connector 128 may include the septum 206, which may be configured to receive the cannula 210 of the housing 204. In some embodiments, the septum 206 and/or the slit may include dimensions such that the cannula 210 extends through the septum 206. In some embodiments, a distal tip 714 of the cannula 210 may be positioned proximate to the distal end 138 of the connector 128. In some embodiments, such positioning of the distal tip 714 may facilitate an effective flushing process by reducing space within the connector 128. In some embodiments, the distal tip 714 may include a bevel 226 having an angle to increase fluid flow through the distal tip 714.

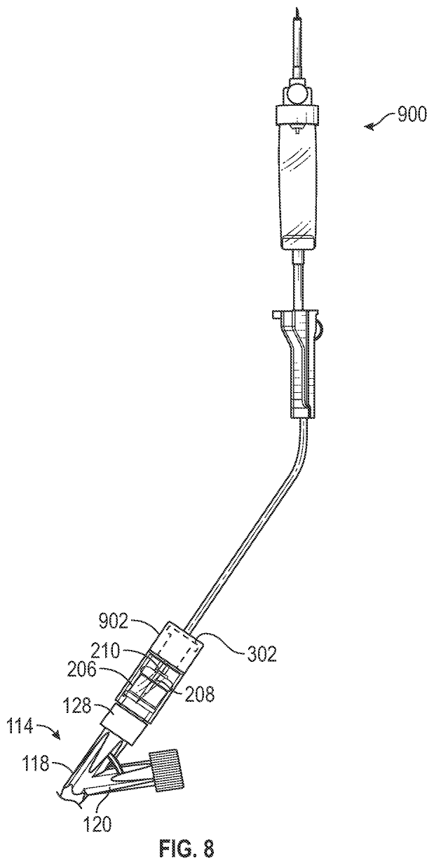

Referring now to FIG. 8, in some embodiments, the needle assembly 202 may be uncoupled from the housing 204 and an extension set 900 may be coupled to the housing 204. In some embodiments, the extension set 900 may include a drip chamber and/or intravenous bag. In some embodiments, the extension set 900 may be used for flushing or infusion. As previously mentioned, in some embodiments, the housing 204 may include the luer 302, such as, for example, a slip or thread male luer connector or a slip or thread female luer connector. In some embodiments, the luer 302 may be coupled to a corresponding luer connector 902 of the extension set 900.

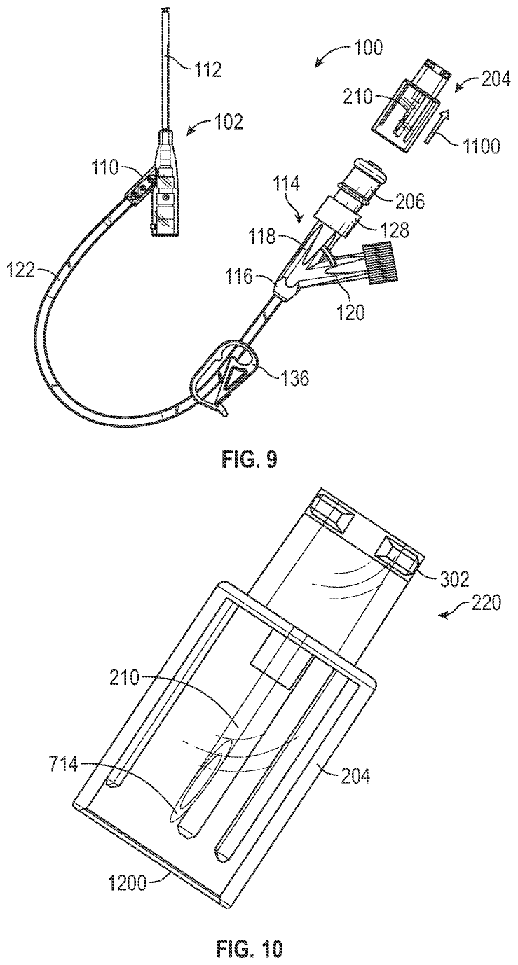

Referring now to FIGS. 9 and 10, following a flush and/or infusion procedure, in some embodiments, the housing 204 may be removed from the connector 128. To this end, in some embodiments, the pinch clamp 136 may be engaged to clamp the extension tube 122 and thereby close the fluid path. Some embodiments of the needle assembly 202 may be removed by applying force to the needle assembly 202 in a proximal direction 1100 relative to the connector 128. In other embodiments, the needle assembly 202 may be removed by disengaging the threading or other coupling feature from the connector 128.

As illustrated in FIG. 10, in some embodiments, the distal tip 714 of the cannula 210 is surrounded or otherwise blocked by the housing 204 to increase safety to the user. In some embodiments, the housing 204 may be a cylindrical shape and may be substantially rigid. In some embodiments, a distal edge 1200 of the housing 204 may extend beyond the distal tip 714 of the cannula 210 to prevent accidental needle stick injury.

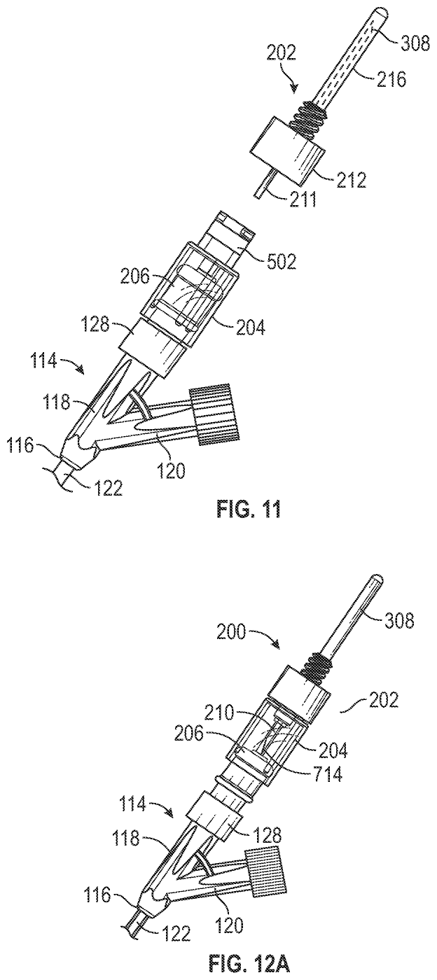

Referring now to FIG. 11, in some embodiments, the connector 128 may include a blood control valve 502 or other suitable blood control technology. In some embodiments, the blood control valve 502 may be disposed within the housing 204 such that, in some embodiments, the pinch clamp 136 may not be needed. Indeed, in some embodiments, the blood control valve 502 may render blood collection, flushing, and/or infusion processes more efficient by automatically preventing blood exposure when removing the needle assembly 202, as illustrated, for example, in FIG. 11. In these and other embodiments, a cannula 211 may extend distally from the body 212 of the needle assembly 202 and may be part of the needle assembly 202. In some embodiments, the cannula 211 may be configured to extend through the blood control valve 502 in response to coupling the needle assembly 202 to the housing 204. In some embodiments, the cannula 211 may include or correspond to the cannula 210 (see, for example, FIGS. 1-10) in terms of one or more features and/or operation. In some embodiments, the cannula 211 may extend through the septum 206. In some embodiments, the cannula 211 may extend through the blood control valve 502 and/or the cannula 210 may extend through the septum 206.

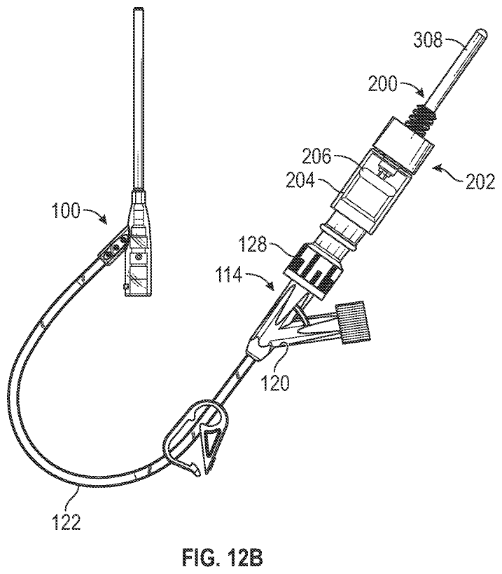

Referring now to FIGS. 12A-12B, in some embodiments, the housing 204 may be coupled to the connector 128 in a proximal position in which the distal tip 714 of the cannula 210 is in close proximity to or contacting the connector 128 without extending through or puncturing the connector 128 or septum 206, as illustrated, for example, in FIGS. 12A. In these and other embodiments, the distal tip 714 may be proximal to the septum 206. In this manner, some embodiments of the catheter system 100 may reduce a risk of leakage by maintaining a closed fluid path during a shelf life of the catheter system 100. In some embodiments, the housing 204 may be pre-attached to the connector 128 in the proximal position. In other embodiments, the housing 204 may be pre-attached to the connector 128 with the cannula 210 extending through the septum 206 of the connector 128.

In some embodiments, the housing 204 may be movable from the proximal position to a distal position, illustrated, for example, in FIG. 12B. In some embodiments, in response to the housing 204 being in the distal position, a fluid path through the septum 206 may be opened. In some embodiments, the user may move the housing 204 from the proximal position to the distal position to push the cannula 210 into and/or through the septum 206 prior to blood collection, for example. In other embodiments, the blood collection set 200 may be packaged together with the catheter system 100 but may not be assembled together or attached until ready for use.

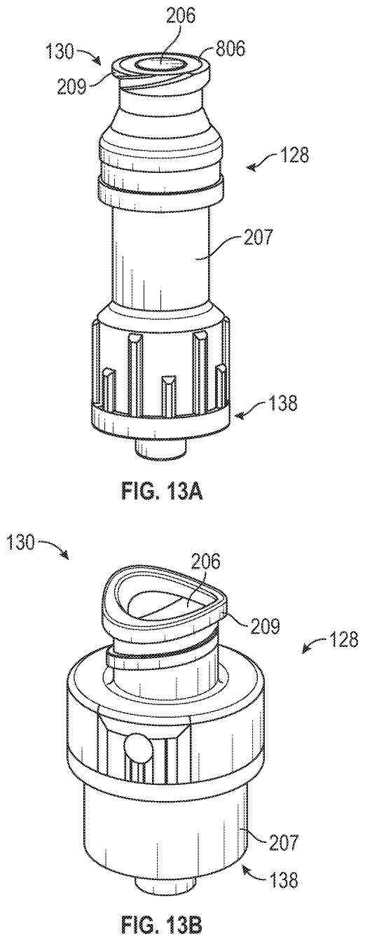

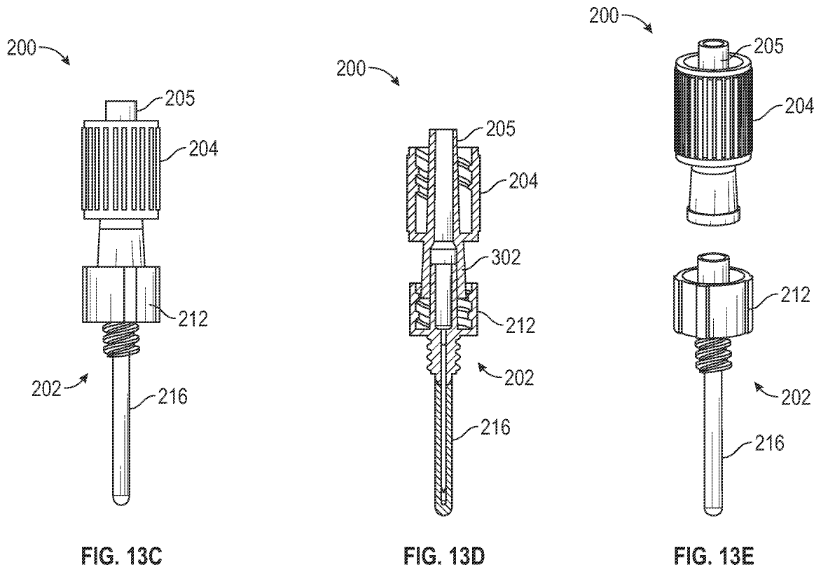

Referring now to FIGS. 13A-13B, in some embodiments, the connector 128 may include a needleless connector. In some embodiments, the connector 128 may include, for example, a MAXPLUS™ needleless connector, a MAXZERO™ needleless connector, a BD Q-SYTE™ luer activated split septum, (available from Becton, Dickinson and Company of Franklin Lakes, New Jersey) a SMARTSITE™ needle-free connector (available from Becton, Dickinson and Company of Franklin Lakes, New Jersey) or another suitable connector. In some embodiments, the needleless connector within the catheter system 100 may not be penetrated by the cannula 210 but may be opened via a male luer or another suitable mechanism.

In some embodiments, the connector 128 may facilitate a flush process by including a proximal end surface 806, which may be substantially flat and/or smooth to avoid collection of residual blood and fluid. In some embodiments, the septum 206 may be disposed within the body 207 of the connector 128. In some embodiments, the proximal end surface 806 may interface with or contact the syringe 802. In some embodiments, the connector 128 may include a proximal end 130 and a distal end 138.

Referring now to FIGS. 13C-13E, the blood collection set 200 is illustrated, according to some embodiments. In some embodiments, the blood collection set 200 may be similar or identical to the blood collection set 200 described, for example, with respect to FIGS. 1-12 in terms of one or more features and/or operation. In some embodiments, the blood collection set 200 may include a housing 204. In some embodiments, the housing 204 may include a male luer 205, which may be configured to couple to the proximal end 130 of the needleless connector, illustrated, for example, in FIGS. 13A-13B. In some embodiments, the male luer 205 may be tapered and/or monolithically formed as a single unit with a remaining portion of the housing 203. In some embodiments, the housing 203 may be removably coupled to the needle assembly 202 or monolithically formed with the needle assembly 202 as a single unit.

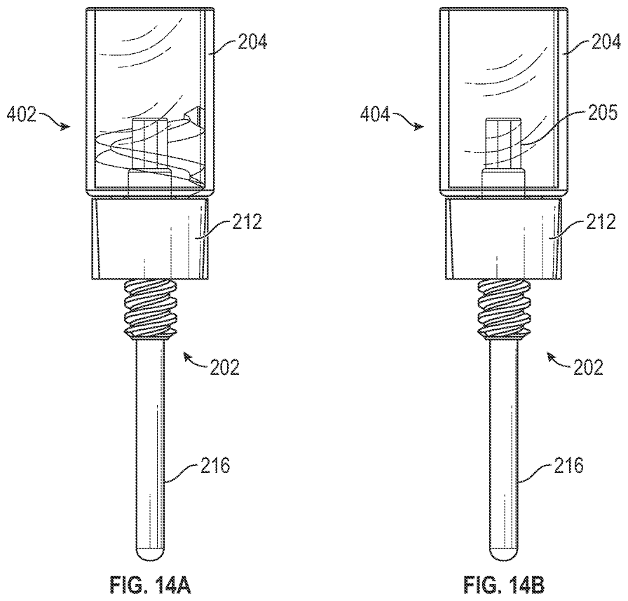

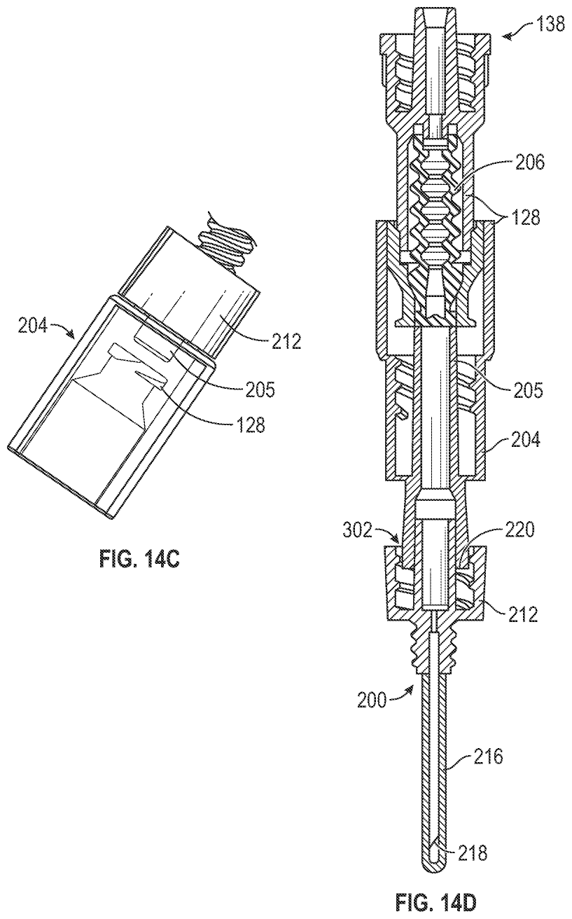

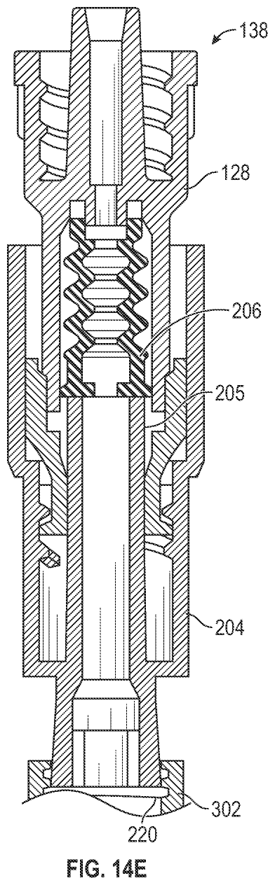

Referring now to FIGS. 14A-14B, in some embodiments, the housing 204 may include the luer 302 (see, for example, FIG. 2B) and/or a male luer 205, which may be disposed on an inner surface of the housing 204. In some embodiments, the male luer 205 may include a luer lock 402 or a luer slip 404. In some embodiments, a protrusion of the male luer 205 may extend beyond the cylindrical portion of the housing 204 as illustrated, for example, in FIGS. 13C-13E. In other embodiments, the protrusion of the male luer 205 may not extend beyond the cylindrical portion of the housing 204 and may be disposed within the cylindrical portion. In some embodiments, the male luer 205 may be configured to engage a luer 209 (see, for example, FIGS. 13A-13B) at the proximal end 130 of the needleless connector.

Referring now to FIGS. 14C-14E, in some embodiments, the housing 204 may be disposed in a proximal position illustrated, for example, in FIG. 14D. In these embodiments, the connector 128 may be coupled to the male luer 205 of the housing 204 such that a the male luer 205 is maintained adjacent to, but not inserted into, the connector 128. As illustrated, in FIG. 14C, in some embodiments, the male luer 205 and the connector 128 may be spaced apart.

In some embodiments, the housing 204 and/or the needle assembly 202 may be movable from the proximal position to a distal position, illustrated, for example, in FIG. 14E. In some embodiments, in response to the housing 204 being in the distal position, the male luer 205 may be fully inserted into the connector 128. In some embodiments, the housing 204 may be moved from the proximal position to the distal position by the user prior to performing a blood collection procedure.

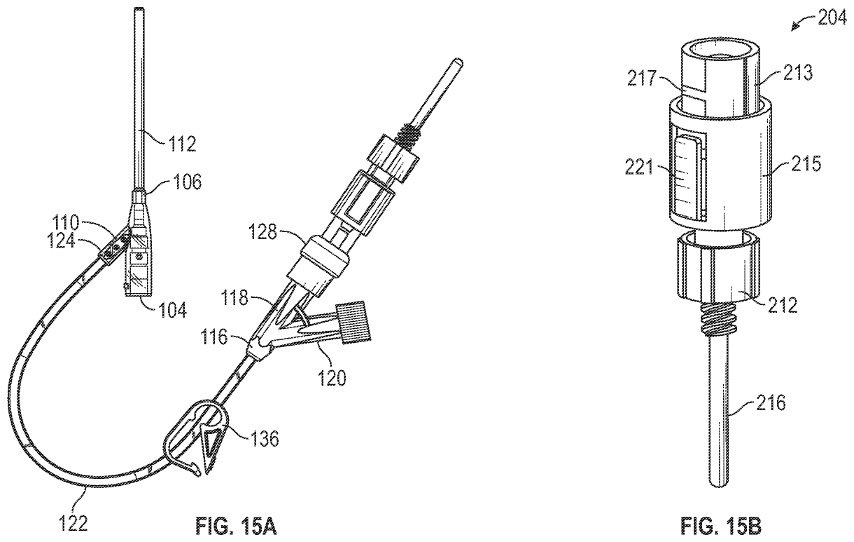

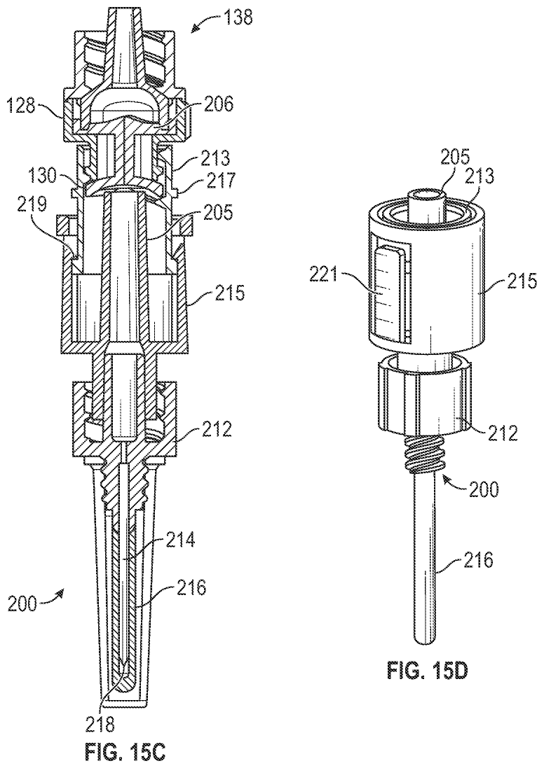

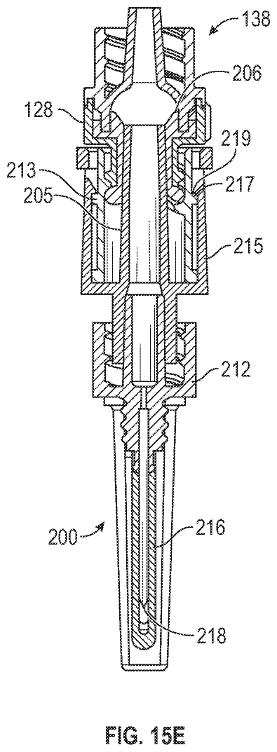

Referring now to FIGS. 15A-15E, in some embodiments, the housing 204 may include a distal piece 213 and a proximal piece 215 movable distally towards the distal piece 213. In some embodiments, the distal piece 213 of the housing 204 may include the male luer 205. As illustrated, for example, in FIGS. 15D-15E, in some embodiments, the proximal piece 215 may be moved distally towards the distal piece 213. In some embodiments, the male luer 205 may activate the septum 206 and create the fluid path through the connector 128 in response to the proximal piece 215 moving distally towards the distal piece 213. In some embodiments, an outer surface of the distal piece 213 may include one or more protrusions 217, which may facilitate securement of the proximal piece 215 in a proximal position, illustrated, for example, in FIG. 15C, and/or a distal position, illustrated, for example, in FIG. 15E. In some embodiments, an inner surface of the housing 204 may include one or more protrusions 219 configured to slide past the protrusions 217 and engage the proximal piece 215 in a snap-fit with the distal piece 213. In some embodiments, the protrusions 219 may be disposed on an inner surface of an arm 221 of the proximal piece 215 of the housing 204, which may be detached from a remaining portion of the proximal piece 215 along opposing edges and an end of the arm 221 to provide flexing.

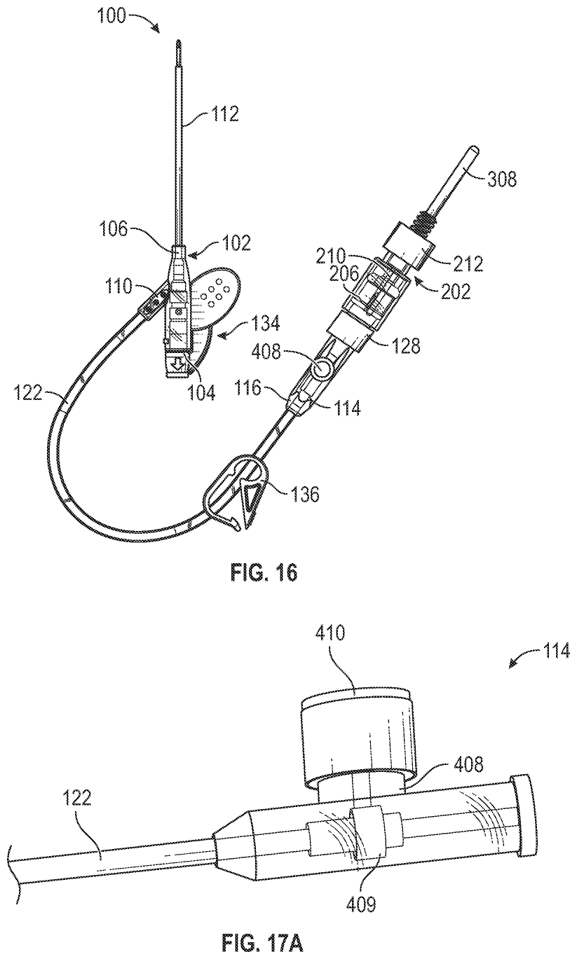

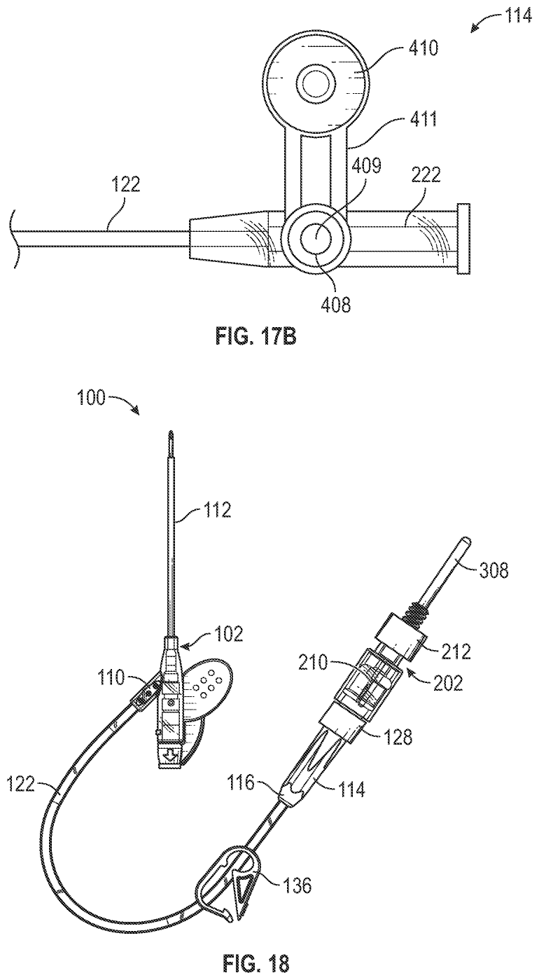

Referring now to FIGS. 16, 17A and 17B, in some embodiments, the adapter 114 may include an injection port 408 configured to receive the syringe 802 (see, for example, FIG. 5) or other suitable infusion or collection device. In some embodiments, the adapter 114 may be straight, as illustrated, or the adapter 114 may include a Y-adapter or another suitable shape. In some embodiments, the connector 128 may be needleless or configured to receive the cannula 210. In some embodiments, the adapter 114 may include a blood control valve 409 disposed within a lumen 222 of the adapter 114 and which may seal the injection port 408.

In some embodiments, an injection port cap 410 may be coupled to the injection port 408 to avoid contamination of the injection port 408 and/or the blood control valve 409. Some embodiments of the injection port cap 410 may include a hinge 411 or other suitable connection device to maintain the injection port cap 410 in close proximity to the injection port 408. Some embodiments of the injection port cap 410 may include dimensions and securement features to facilitate easy opening and secure closing of the injection port 408. In some embodiments, the injection port cap 410 may be press fit to the injection port 408 to selectively close access to the adapter 114. Referring now to FIG. 18, the adapter 114 is illustrated in a straight configuration and without the injection port 408, according to some embodiments.

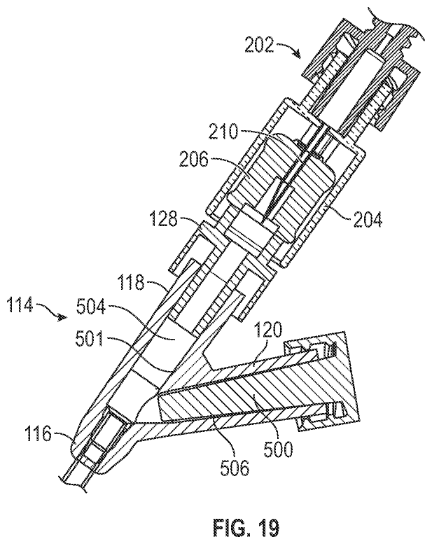

Referring now to FIG. 19, in some embodiments, a particular port of the adapter 114, such as the second port 120 of the adapter 114, may include an elongate end cap 500. In some embodiments, the elongate end cap 500 may extend even with or distal to an inner wall 501 forming a lumen 504 of the first port 118. In this manner, some embodiments of the adapter 114 may reduce blood contamination of the second port 120. In some embodiments, the elongate end cap 500 may be disposed within a lumen 506 of the second port 120. In some embodiments, the connector 128 may be needleless or configured to receive the cannula 210.

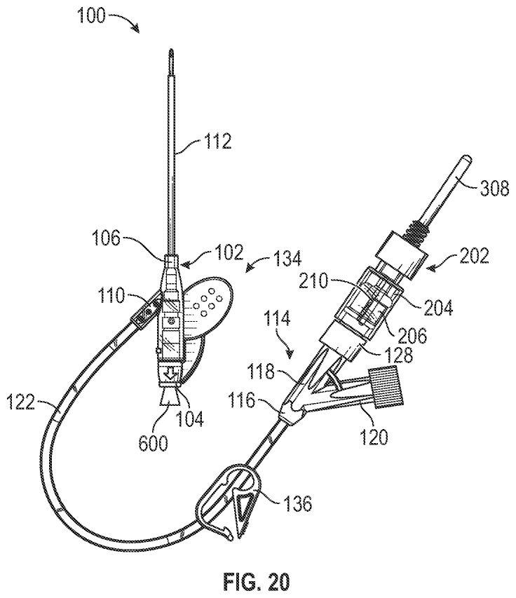

Referring now to FIG. 20, in some embodiments, the catheter system 100 may include a secondary flashback chamber 600 disposed at the proximal end 104 of the catheter adapter 102. In operation, in some embodiments, placement of the catheter 112 within the vasculature may be confirmed by blood or “instaflash” visible within the catheter 112 and/or a primary flashback chamber within the catheter adapter 102. In some embodiments, the secondary flashback chamber 600 may facilitate observance of blood in the secondary flashback chamber 600 to enhance initial vein confirmation, as well as continued placement of the catheter 112 within the vein during blood collection, flushing, or infusion.

In some embodiments, in response to confirmation of proper placement of the catheter 112 within the vein, the needle assembly 202 and/or the housing 204 of the blood collection set 200 may be engaged with the connector 128 to open a fluid path for blood collection. After blood collection, in some embodiments, the needle assembly 202 may be removed and the syringe 802 may be coupled to the housing 204 for flushing of the catheter system 100 or for infusion of drugs, saline, or the like. In some embodiments, the connector 128 may be needleless or configured to receive the cannula 210.

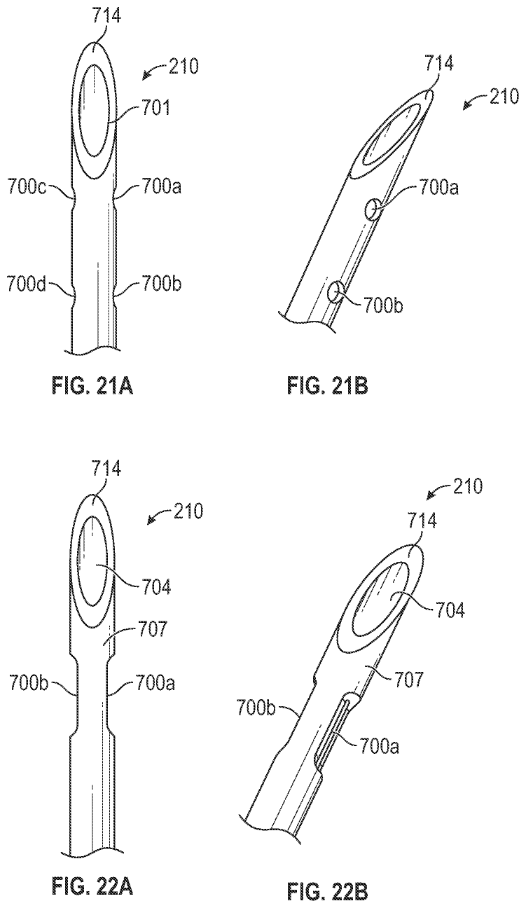

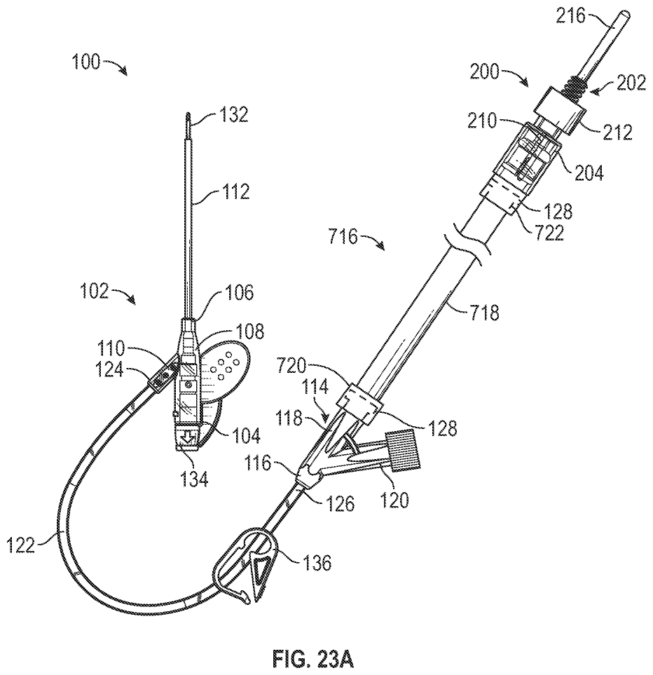

Referring now to FIG. 21A-21B and 22A-22B, in some embodiments, the cannula 210 may include one or more side holes 700 which, along with an opening 701 in the distal tip 714, provide multiple fluid paths through the distal tip 714. In some embodiments, the side holes 700 may be disposed near the distal tip 714. In some embodiments, the side holes 700 and/or the opening 701 may be in fluid communication with a lumen of the cannula 210, which may extend through the cannula 210. In some embodiments, the septum 206 (see, for example, FIG. 2B) may be spaced apart from the side holes 700 to avoid occlusion of the side holes 700 in response to the cannula 210 extending through the septum 206.

In some embodiments, as illustrated in FIGS. 21A-21B, the side holes 700 may be substantially annular and may be disposed in one or more sides of the cannula 210. In some embodiments, the side holes 700 may be disposed on opposite sides of the cannula 210. In some embodiments, one or more of the side holes 700 may be formed to extend through the side of the cannula 210 at an angle. In some embodiments, each of the side holes 700 may be substantially uniform or non-uniform relative to one another. In these and other embodiments, the cannula 210 may include any number of side holes 700, and each of the side holes 700 may include any suitable dimension, shape and/or orientation.

As illustrated in FIGS. 22A-22B, for example, one or more of the side holes 700 may include a substantially orthogonal shape. In some embodiments, at least one of the side holes 700 may be formed as a slit extending through a side 707 of the cannula 210 to provide, together with the distal tip 714, multiple routes of fluid communication between a lumen 704 of the cannula 210 and an exterior environment.

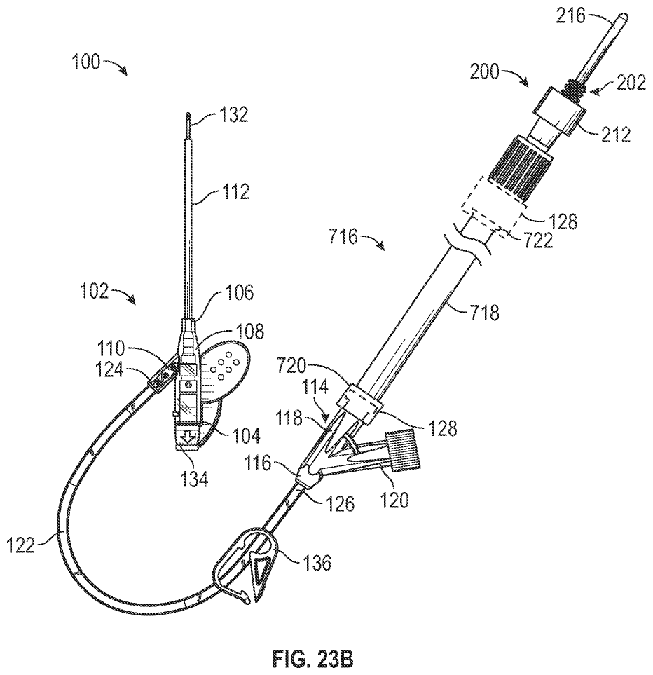

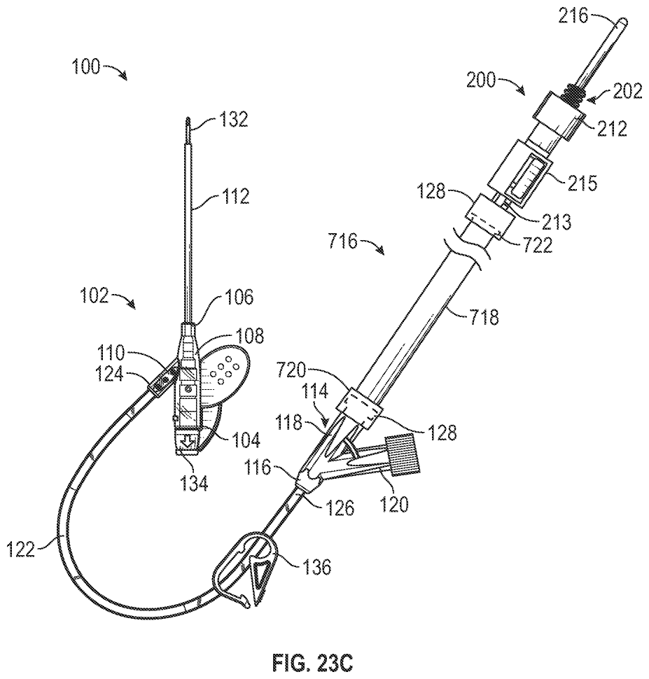

Referring now to FIGS. 23A, in some embodiments, the connector 128 may be coupled to the adapter 114, and the blood collection set 200 may be coupled to the connector 128. In other embodiments, as illustrated, for example, in FIG. 23, the connector 128 may be coupled to a proximal end of an extension set 716. In some embodiments, the extension set 716 may include an extension tube 718, which may include a distal end integrated within a distal luer 720 and/or a proximal end integrated within a proximal luer 722. In some embodiments, the blood collection set 200 may be coupled the connector 128. Referring now to FIGS. 23B-23C, in some embodiments, various configurations of the blood collection set 200 described with respect to one or more of FIGS. 1-23 may be disposed at the proximal end of the extension set 716.

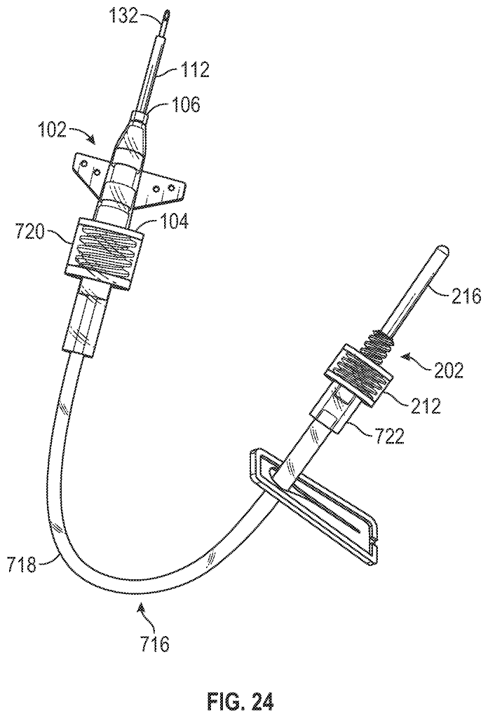

Referring now to FIG. 24, in some embodiments, the extension set 716 may be coupled to the proximal end 104 of the catheter adapter 102, and the needle assembly 202 may be coupled to the proximal end of the extension tube 718. In some embodiments, the proximal end of the extension tube 718 may be integrated within the needle assembly 202. In other embodiments, the needle assembly 202 may be removably coupled to the proximal luer 722. In some embodiments, various configurations of the blood collection set 200 described with respect to one or more of FIGS. 1-23 may be disposed at the proximal end of the extension set 716.

In some embodiments, the catheter adapter 102 may include an open catheter adapter, which may not include an integrated extension tube. In some embodiments, the extension set 716 may be coupled to the proximal end 104 of the catheter adapter 102. In some embodiments, the needle assembly 202 may be pre-attached to the extension set 716 or coupled to the extension set 716 prior to coupling of the extension set 716 to the proximal end 104 of the catheter adapter 102.

In some embodiments, after placement of the catheter 112 within the vasculature of the patient, the blood collection device 306, which may include the blood collection tube, may be coupled to the needle assembly 202, and blood may be collected within the blood collection device 306. In some embodiments, in response to the blood collection device 306 pushing the sheath 216 distally towards the body 212, the sharp needle 214 may pierce the sheath 216 and the sharp proximal tip 218 of the needle assembly 202 may be inserted into the blood collection device 306 for blood collection.

In some embodiments, after collecting the blood, the pinch clamp 136 may be engaged to stop fluid flow through the fluid path of the catheter system 100, which may extend from the catheter 112, through the catheter adapter 102, through the extension set 716, and into the needle assembly 202. In some embodiments, after engaging the pinch clamp 136, the needle assembly 202 may be uncoupled from the extension set 176 and removed from the catheter system 100. In some embodiments, after removal of the needle assembly 202, the proximal end of the extension set 716 may then be coupled to a flush or infusion device. In some embodiments, after coupling the proximal end of the extension set 716 to the flush or infusion device, the pinch clamp 136 may be disengaged and the fluid path may be opened, followed by flushing or infusion into the vasculature of the patient through the extension set 716 and the catheter adapter 102 coupled to the extension set 716.

In some embodiments, after flush or infusion, the pinch clamp 136 may be engaged to stop flow through the extension set 176 and/or the needle assembly 202 may then be coupled to a connector, such as, for example, the connector 128 (see one or more of FIGS. 1-20). In some embodiments, the connector 128 may be coupled to the proximal end of the extension set 176 and/or the housing 204 (see, for example, one or more of FIGS. 1-20) may be coupled to the connector 128. In these and other embodiments, the housing 204 may be uncoupled from the connector 128 and removed from the catheter system 100 after flush or infusion via the flush or infusion device coupled to the housing 204.

All examples and conditional language recited herein are intended for pedagogical objects to aid the reader in understanding the present disclosure and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

1. A catheter system, comprising:
a catheter adapter, comprising a distal end, a proximal end, and a lumen extending through the distal end of the catheter adapter and the proximal end of the catheter adapter;
a catheter extending distally from the distal end of the catheter adapter;
a connector coupled to the catheter adapter and comprising a septum; and
a blood collection set, comprising a needle assembly and a housing, wherein the housing is coupled to the connector, wherein the housing is configured to create a fluid path through the connector.

2. The catheter system of claim 1, wherein the housing is directly and removably coupled to the needle assembly.

3. The catheter system of claim 1, wherein the housing is integrated with the needle assembly.

4. The catheter system of claim 1, further comprising:
a side port disposed between the distal end of the catheter adapter and the proximal end of the catheter adapter;
an extension tube, comprising a distal end integrated with the side port of the catheter adapter and a proximal end integrated with the distal end of the adapter; and
an adapter, comprising a distal end and a port, wherein the connector is coupled to the port of the adapter.

5. The catheter system of claim 4, wherein the adapter comprises a Y-adapter or a straight adapter.

6. The catheter system of claim 1, wherein the connector is a needleless connector.

7. The catheter system of claim 6, wherein the housing is disposed in a proximal position, wherein the housing comprises a male luer, wherein the male luer is configured to activate the septum and create the fluid path through the connector in response to the housing being moved from the proximal position to a distal position, wherein the housing is threaded from the proximal position to the distal position or pushed from the proximal position to the distal position.

8. The catheter system of claim 6, wherein the housing comprises a distal piece and a proximal piece movable distally towards the distal piece, wherein the distal piece of the housing comprises a male luer, wherein the male luer is configured to activate the septum and create the fluid path through the connector in response to the proximal piece moving distally towards the distal piece.

9. The catheter system of claim 1, wherein the housing comprises a cannula configured to penetrate the septum of the connector to create the fluid path through the connector.

10. The catheter system of claim 9, wherein the cannula comprises a side hole in fluid communication with a lumen of the cannula.

11. The catheter system of claim 9, wherein the housing is movable from a proximal position to a distal position, wherein in response to the housing moving from the proximal position to the distal position, the cannula extends through the septum and the fluid path through the connector is created.

12. The catheter system of claim 9, wherein the cannula extends through the septum.

13. The catheter system of claim 1, further comprising an extension set, wherein the extension set comprises a distal end coupled to the proximal end of the catheter adapter and a proximal end coupled to the connector, wherein the connector is a needleless connector.

14. A system, comprising:
an extension set, wherein the extension set comprises:
a distal end configured to couple to a proximal end of the catheter adapter; and
a proximal end; and
a needle assembly coupled to the proximal end of the extension set, wherein the needle assembly comprises a body, a sharp needle extending proximally from the body, and a sheath covering a proximal tip of the sharp needle.

15. The system of claim 14, wherein the needle assembly is directly coupled to the proximal end of the extension set.

16. The system of claim 14, further comprising:
a needleless connector directly coupled to the proximal end of the extension set, wherein the needleless connector comprises a septum; and
a housing directly coupled to the needleless connector and the needle assembly, wherein the housing is configured to create a fluid path through the needleless connector.

17. The system of claim 14, further comprising:
a catheter adapter, comprising a distal end, a proximal end, and a lumen extending through the distal end of the catheter adapter and the proximal end of the catheter adapter; and
a catheter extending distally from the distal end of the catheter adapter.

18. A blood collection set to couple to a catheter system, comprising:
a needle assembly, comprising a body, a sharp needle extending proximally from the body, and a sheath covering a proximal tip of the sharp needle; and
a housing coupled to a distal end of the needle assembly, wherein the housing is configured to create a fluid path through a connector.

19. The blood collection set of claim 18, wherein the housing is directly and removably coupled to the needle assembly.

20. The blood collection set of claim 18, wherein the housing is integrated with the needle assembly.

21. The blood collection set of claim 18, wherein a distal end of the housing comprises a male luer, wherein the male luer comprises threading or is configured for a slip fit.

22. The blood collection set of claim 18, wherein the housing comprises a distal piece and a proximal piece movable distally towards the distal piece, wherein the distal piece of the housing comprises a male luer, wherein the male luer is configured to activate a septum of the connector and create the fluid path through the connector in response to the proximal piece moving distally towards the distal piece.

23.-33. (canceled)
